(19) United States
(12) Patent Application Publication
(10) Pub. No.: US 2018/0014908 A1
(43) Pub. Date: Jan. 18, 2018

(54) TROCAR DEVICE

(71) Applicants: BAY PATH UNIVERSITY, Longmeadow, MA (US); Western New England University, Springfield, MA (US)

(72) Inventors: Brian Katz, Longmeadow, MA (US); Robert Gettens, Longmeadow, MA (US); Mary G. Schoonmaker, Longmeadow, MA (US); Nicolette R. LaPierre, Windsor Locks, CT (US); Shaine Gregory Spencer, Tewksbury, MA (US); Samuel Thomas Mackerer, Ghent, NY (US); Marc Castiello, Derby, CT (US); Trinh Thuy Pham, Springfield, MA (US)

(21) Appl. No.: 15/642,851

(22) Filed: Jul. 6, 2017

Related U.S. Application Data
(60) Provisional application No. 62/361,215, filed on Jul. 12, 2016.

(51) Int. Cl. A61B 17/34; A61B 1/12

(52) U.S. Cl. CPC A61B 90/70; A61B 17/3498; A61B 1/126; A61B 17/3423; A61B 2017/3437

(57) ABSTRACT

A trocar device is provided. The trocar includes a body having a first passageway sized to receive a surgical instrument. A cannula is coupled to the body, the cannula having a second passageway that cooperates with the first passageway to allow the surgical instrument to pass therethrough. A plurality of orifices is operably coupled to one of the body or the cannula and arranged to direct a washing fluid onto at least a portion of the surgical instrument in one of the first passageway or the second passageway. An output port is fluidly coupled to receive the washing fluid from one of the first passageway or second passageway.

### CROSS-REFERENCE TO RELATED APPLICATIONS

The present application is a Nonprovisional application of U.S. Provisional Application Ser. No. 62/361,215 filed on Jul. 12, 2017, the contents of which is incorporated by reference in its entirety.

### BACKGROUND

The subject matter disclosed herein relates to a trocar device, and in particular to trocar device including an integrated cleaning system for washing surgical devices.

Minimally invasive surgeries have become a common method of surgery in recent years. One of the most common minimally invasive surgeries is laparoscopic surgery. Laparoscopic surgery is a method used to perform procedures within the abdominal or pelvic regions of the patient's body. Nearly, 2.5 million laparoscopic procedures are performed annually in the United States. Laparoscopic surgeries use a scope and lens to visualize the procedure through a slight incision. During the procedure, the scope usually gets covered in bodily fluids, reducing visibility for the surgeon. Current methods on the market to limit this problem are defogging devices or sprays used prior to the scope being initially inserted into the patient's body. However, these methods have been inefficient and generally not effective in maintaining a desired level of visualization for the surgeon.

Accordingly, while existing surgical implements are suitable for their intended purpose the need for improvement remains, particularly in providing an integrated biocompatible cleaning arrangement that allows bodily fluids to be removed from the scope during the surgical procedure.

### BRIEF DESCRIPTION

According to one aspect of the disclosure a trocar device is provided. The trocar includes a body having a first passageway sized to receive a surgical instrument. A cannula is coupled to the body, the cannula having a second passageway that cooperates with the first passageway to allow the surgical instrument to pass therethrough. A plurality of orifices is operably coupled to one of the body or the cannula and arranged to direct a washing fluid onto at least a portion of the surgical instrument in one of the first passageway or the second passageway. An output port is fluidly coupled to receive the washing fluid from one of the first passageway or second passageway.

According to another aspect of the disclosure a trocar device is provided. The trocar device includes a body having a first passageway sized to receive a surgical instrument. A cannula is coupled to the body, the cannula having a second passageway that cooperates with the first passageway to allow the surgical instrument to pass therethrough. A turbine cleaning wheel is rotationally coupled to the body, the turbine cleaning wheel having a first opening sized to receive the surgical instrument. An input port is arranged to direct a washing fluid onto the turbine cleaning wheel. An output port is fluidly coupled to receive the washing fluid from the first passageway.

According to yet another aspect of the disclosure a method of cleaning a surgical instrument during a surgical procedure is provided. The method includes providing a trocar device in an incision made during the surgical procedure, the trocar device having a body with a first passageway and a cannula with a second passageway. The surgical instrument is inserted into the trocar device. A washing fluid flows into the trocar device via an input port. The washing fluid flows through a plurality of orifices onto the surgical instrument in one of the first passageway or the second passageway. The washing fluid is removed from one of the first passageway or second passageway via an output port.

These and other advantages and features will become more apparent from the following description taken in conjunction with the drawings.

### BRIEF DESCRIPTION OF DRAWINGS

The subject matter, which is regarded as the disclosure, is particularly pointed out and distinctly claimed in the claims at the conclusion of the specification. The foregoing and other features, and advantages of the disclosure are apparent from the following detailed description taken in conjunction with the accompanying drawings in which:

FIG. 1 is a perspective view of a trocar device in a accordance with an embodiment of the invention;

FIG. 2 is an exploded view of the trocar device of FIG. 1;

FIG. 3 is a sectional view through a centerline of the trocar device of FIG. 1;

FIG. 4 is a sectional view of a port member for use with the trocar device of FIG. 1, in accordance with an embodiment of the invention;

FIG. 5 is a partial perspective view of the port member of FIG. 4;

FIG. 6 is a partial perspective view of a top end portion of a cannula for use with the trocar device of FIG. 1, in accordance with an embodiment of the invention;

FIG. 7 is a partial perspective view of an opposite end of the cannula of FIG. 6;

FIG. 8 is an end view of the cannula of FIG. 6;

FIG. 9 is an end view of the port member of FIG. 4;

FIG. 10 is a perspective view of a trocar device in accordance with another embodiment of the invention;

FIG. 11 is a perspective view of a trocar device in accordance with another embodiment of the invention;

FIG. 12 is a side view of a y-tubing adapter to couple the trocar device of FIG. 11 to a suction/aspiration device; and

FIG. 13 is a perspective view of a trocar device in accordance with another embodiment of the invention.

The detailed description explains embodiments of the disclosure, together with advantages and features, by way of example with reference to the drawings.

### DETAILED DESCRIPTION

Embodiments of the present invention provide for a trocar device that is used in surgical procedures. Embodiments of the invention provide for a trocar device that incorporates an integrated cleaning system for cleaning a surgical implement, such as a scope having a lens used in a surgical video system for example. Embodiments of the invention provide for a trocar having an integrated cleaning system that uses a biocompatible cleaning solution that allows for the surgical implements to be cleaned during the surgical procedure without removing the surgical instrument from the trocar device. Embodiments of the present invention are further advantageous in being operable with conventional laparoscopy surgery and with robotic laparoscopic surgery procedures.

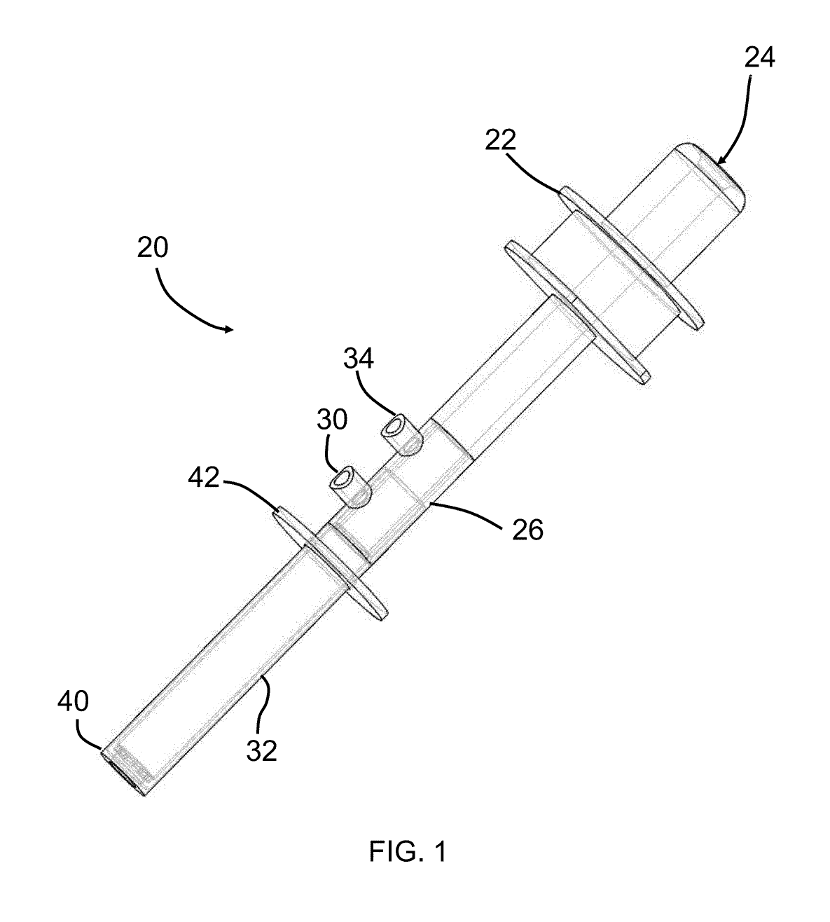

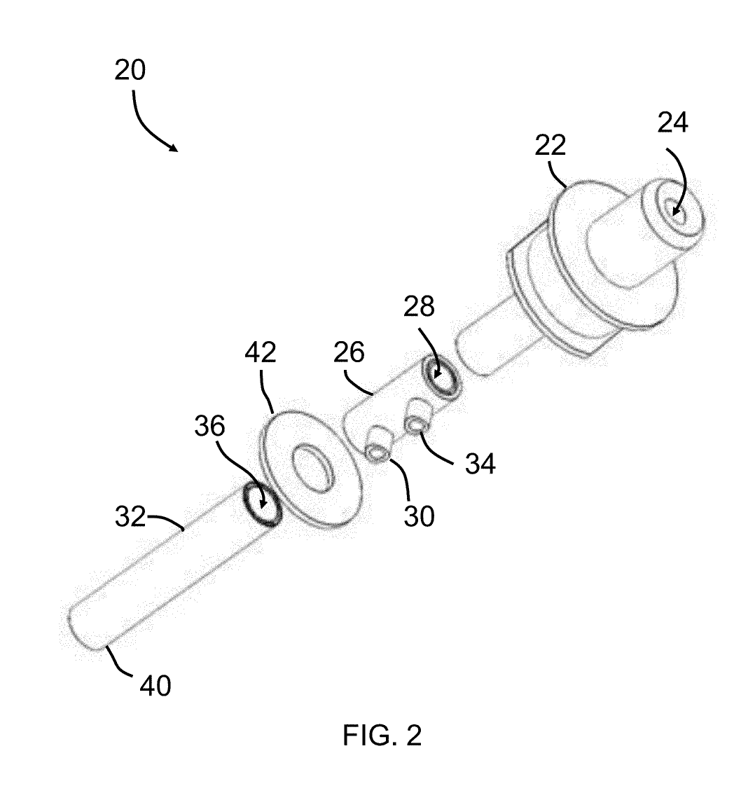

A trocar is a device that is placed within an opening or incision of a patient during surgical procedures. The trocar allows surgical instruments through the incision and also prevents pressure build-up of fluids and gasses. Referring now to FIGS. 1-3, an embodiment is shown of a trocar device 20. The trocar 20 includes a body 22 having an internal passageway 24 that extends therethrough. The internal passageway 24 is sized to receive surgical instruments, such as a scope used in a laparoscopic procedure for example. The body 22 is coupled to a port member 26. As discussed in more detail herein, the port member 26 includes a central bore 28 that is operably coupled to the internal passageway 24 to allow the surgical instruments to pass therethrough. The port member 26 is configured to receive a biocompatible washing fluid via an input port 30 and direct the fluid into a cannula 32. The port member 26 is further configured to couple with an aspiration vacuum device (not show) via an output port 34 for removing the washing fluid from the trocar 20. In the exemplary embodiment, the washing fluid is saline. It should be appreciated that while embodiments herein the washing fluid as being saline, this is for the exemplary purposes and the claimed invention should not be so limited. In other embodiments, other biocompatible fluids may be used.

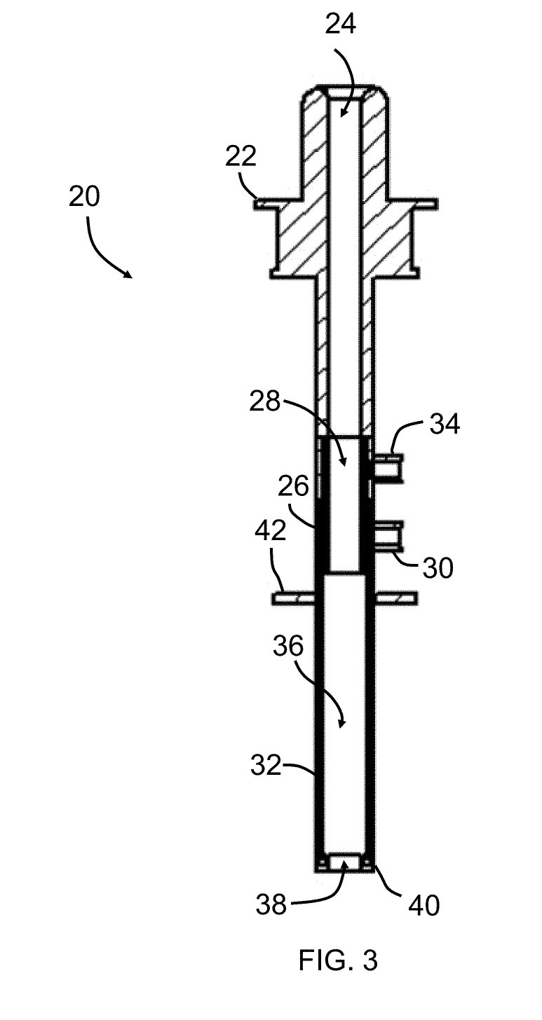

The cannula 32 is a generally cylindrical member having a passageway 36 that is operably coupled to the bore 28 to receive surgical instruments from the passageway 24. The passageway 36 includes an opening 38 (FIG. 3) on an end 40 opposite the port member 26. The opening 38 allows the surgical instruments to pass into the patient during the surgical procedure. In an embodiment, a distal donut member 42 is disposed about the outer diameter of the port member 26 or the end of the cannula 32. The distal donut member 42 is configured to prevent movement of the trocar relative to the patient's body.

In operation, when the surgeon desires to clean the surgical instrument, such as to improve visibility through the end of a lens on a scope for example. The surgeon pulls the instrument into the passageway 36 of the cannula 32. A biocompatible washing fluid, such as but not limited to saline for example, flows through the input port 30 and is directed into the cannula 32. The washing fluid enters the passageway 36 adjacent the end 40 and flows through the passageway 36 back towards the port member 26. As will be discussed in more detail below, ducts and channels within the port member 26 fluidly couple the passageway 36 to the output port 34 to allow the washing solution to be removed from the trocar 20. It should be appreciated that as the washing fluid flows from the end 40 to the port member 26, the bodily fluids contaminating the surgical instrument may be removed and the surgical instrument cleaned.

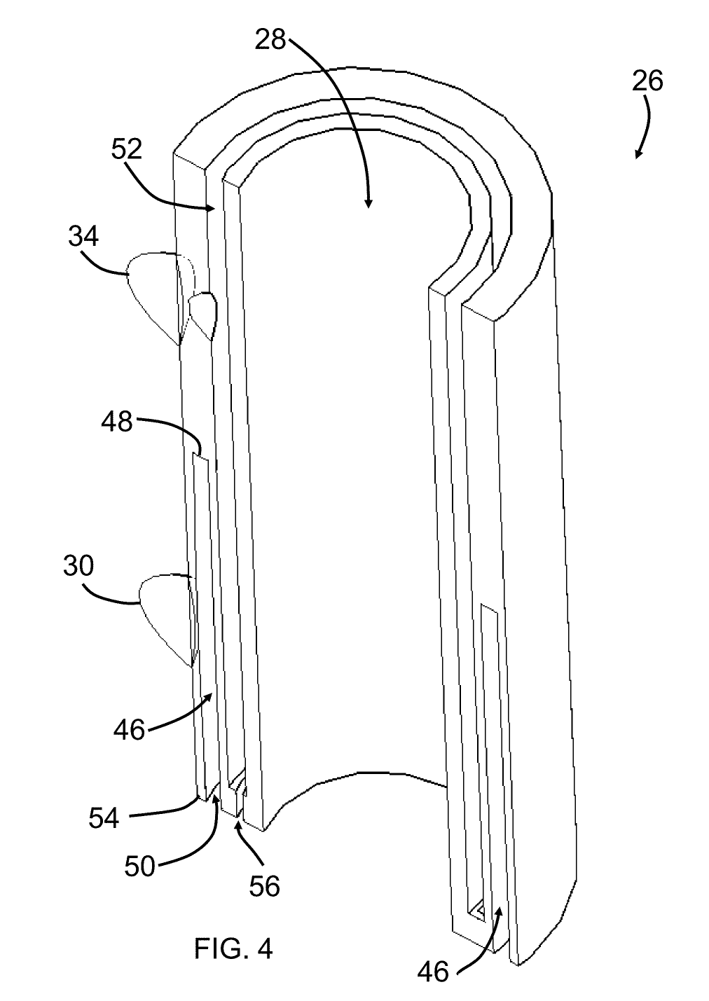

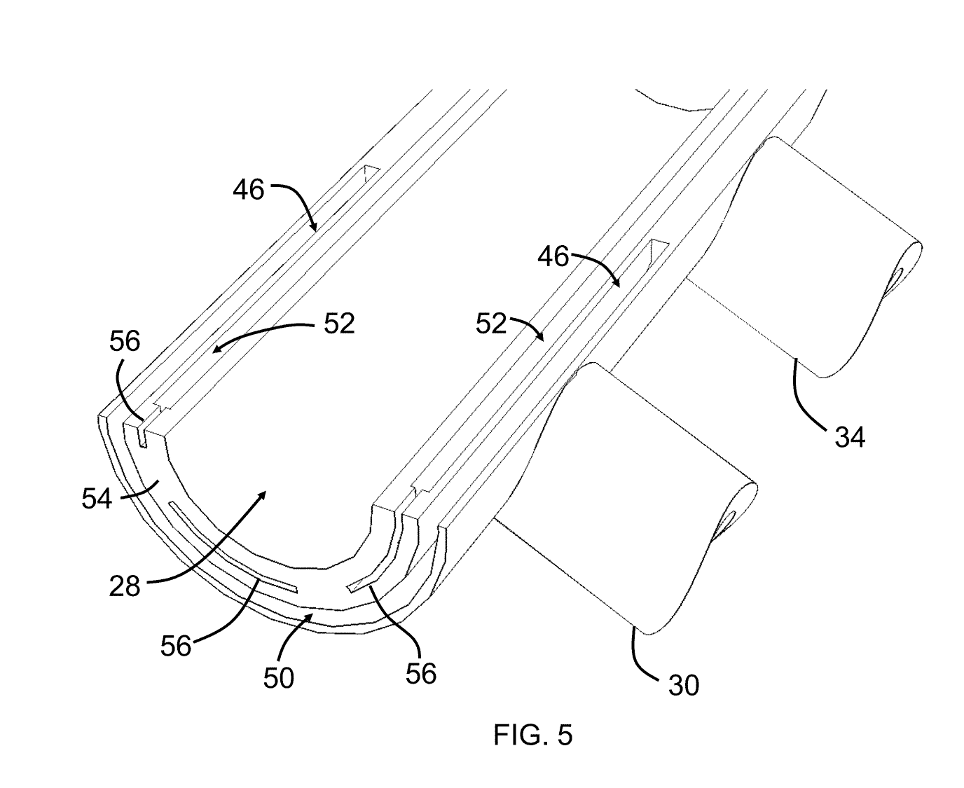

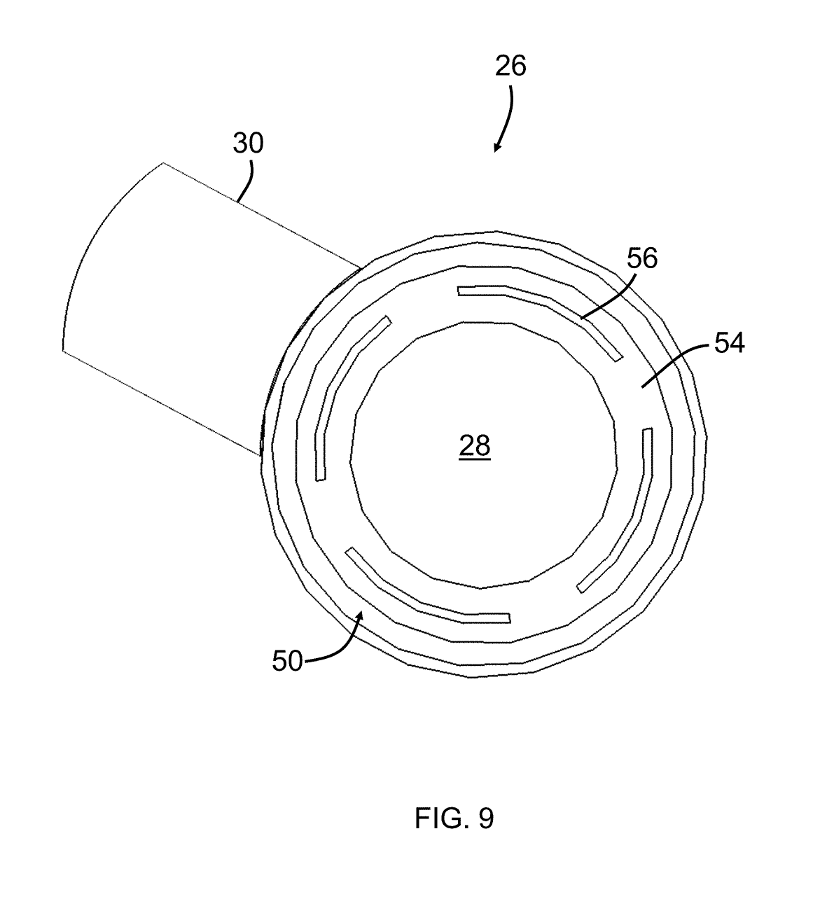

Referring now to FIG. 4, FIG. 5 and FIG. 9, an embodiment is shown of a port member 26. The port member 26 includes an input port 30 that is fluidly coupled to an annular channel 46. The channel 46 extends circumferentially about the port member 26. One end 48 is closed, while an opposing end has an opening 50. As will be discussed in more detail herein, the opening 50 cooperates with a channel in the cannula 32 to flow the washing fluid towards end 40.

The port member 26 further includes an annular duct 52 that is fluidly coupled to the output port 34. In the exemplary embodiment, the duct 52 extends circumferentially about the port member 26 is positioned radially inward from the channel 46. At a first end 54, at least one slot 56 extends from the end 54 to the duct 52. In the exemplary embodiment, the at least one slot 56 includes four slots 56 having an arcuate shape and are equally spaced about the end 54. The slots 56 fluidly couple the duct 52 to the passageway 36 to allow removal of washing fluid from the trocar 20 when a vacuum is applied to the output port 34. In one embodiment, the radial width of the slots 56 is smaller than the radial width of the duct 52.

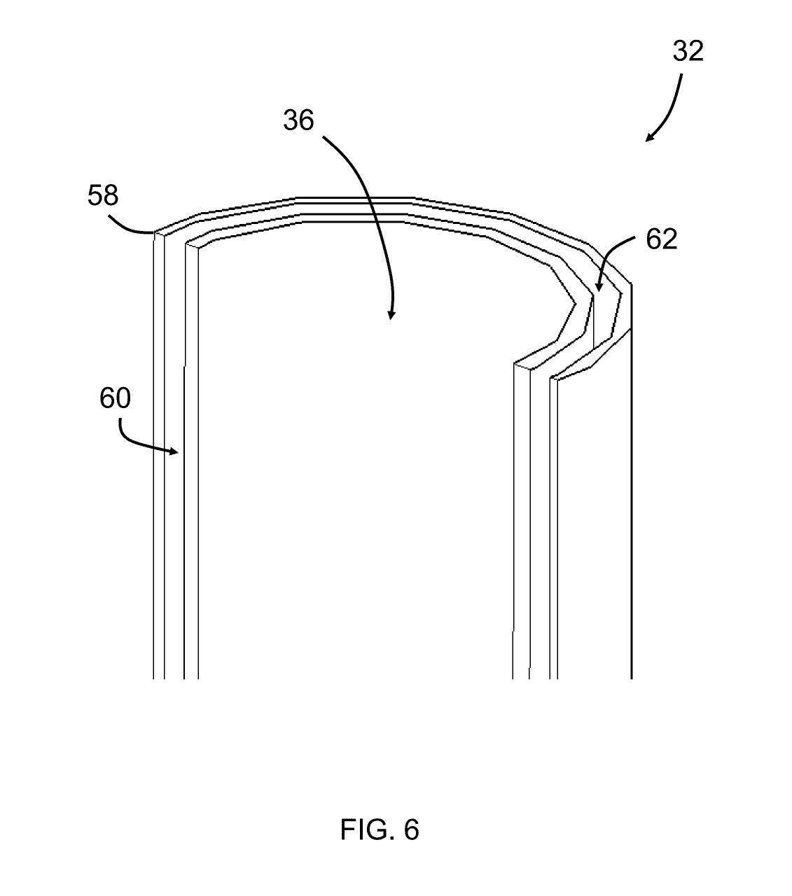

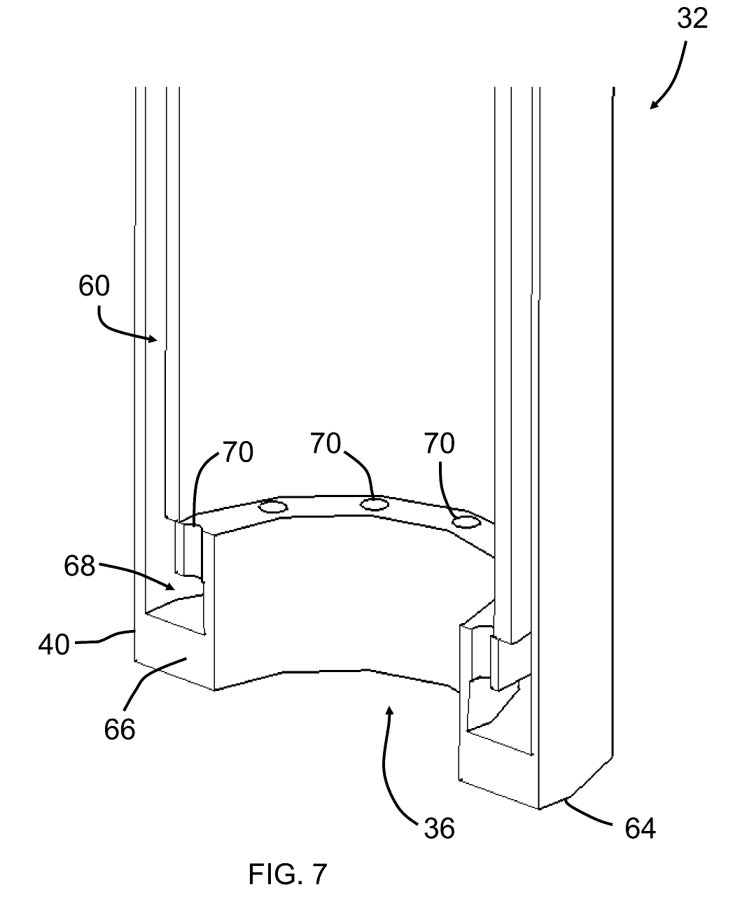

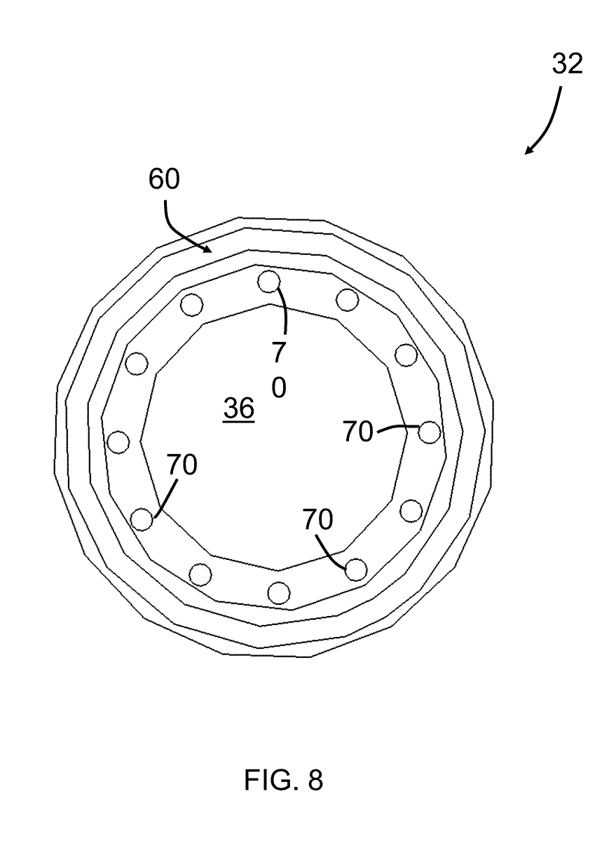

Referring now to FIG. 6, FIG. 7 and FIG. 8, an embodiment is shown of the cannula 32. The cannula 32 is a generally cylindrically shaped body having a first end 58 that couples to the port member 26. The body includes an annular channel 60 having an opening 62 on the end 58. The annular channel 60 traverses the length of the cannula 32. The opening 62 cooperates with and is fluidly coupled to the channel 46 of the port member 26 such that the washing fluid received by the input port 30 flows into the channel 60. At an opposite end 64, the channel 60 terminates in a wall 66. In one embodiment, an annular chamber 68 may be arranged at the end of the channel 60.

A plurality of orifices 70 extends between the chamber 68 and the passageway 36. This allows the washing fluid flowing through the channel 60 to spray into the passageway 36. This spraying of the washing fluid allows the surgical instrument to be cleaned. In the exemplary embodiment, the cannula 32 has 12 orifices disposed equally about the passageway 36. In an embodiment, the passageway 36 has a diameter of 5 mm to 25 mm and the orifices have a diameter of 0.5 mm.

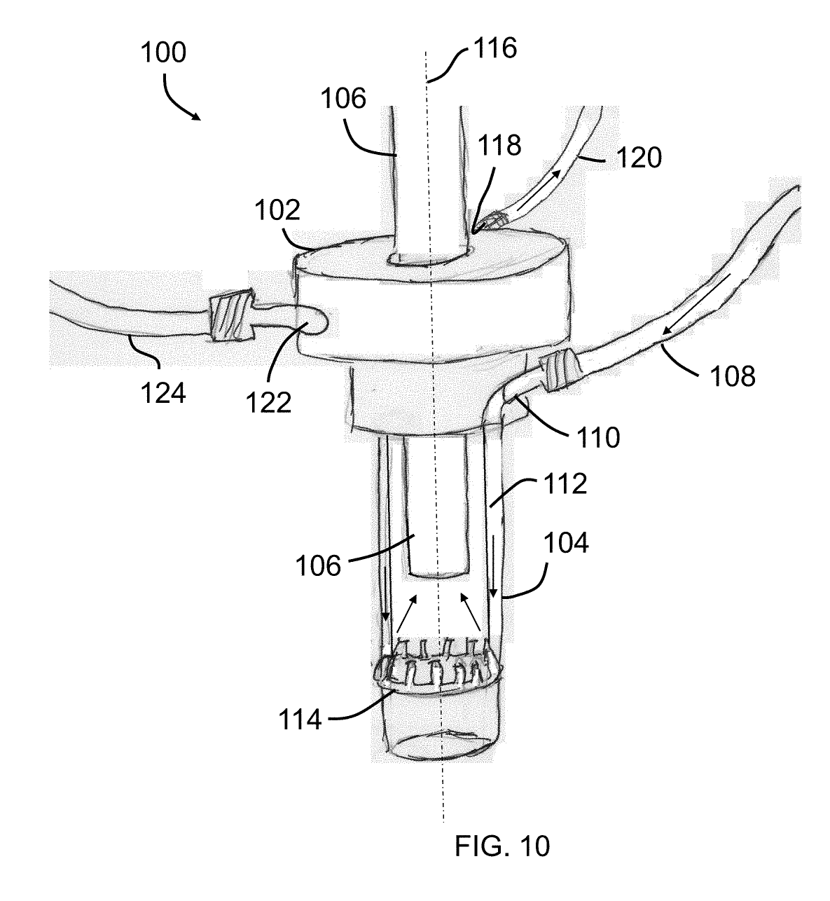

Referring now to FIG. 10 another embodiment is shown of a trocar device 100. The trocar 100 includes a body 102 coupled to a cannula 104. The body 102 and cannula 104 include a internal opening sized to allow a surgical instrument 106 to pass therethrough. A washing solution is received via a conduit 108 through an input port 110. The washing fluid flows through an annular channel 112 to an internal ring of orifices, jets or nozzles 114. The jets 114 are angled to direct the washing fluid towards a centerline 116 of the cannula 104. The inwardly directed washing fluid contacts and cleans the surgical instrument 106. The washing fluid then flows through the cannula 104 towards the body 102 and is withdrawn via an output port 118 and conduit 120. In one embodiment, the trocar 100 includes a gas port 122 to flow CO₂ gas via a conduit 124.

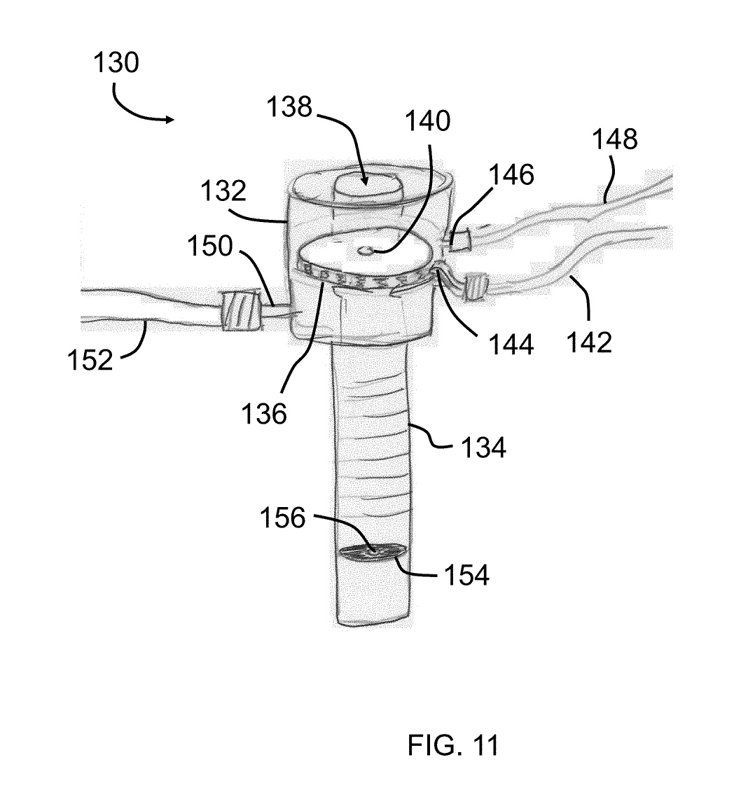

Referring now to FIG. 11, another embodiment is shown of a trocar device 130. In this embodiment, the trocar 130 includes a body 132 having a cannula 134 coupled to one end. A turbine cleaning wheel 136 is rotationally coupled within the body 132. The turbine cleaning wheel 136 is positioned coaxial to the passageway 138 that is sized to receive the surgical instrument. The turbine cleaning wheel 136 includes an aperture or opening 140 having a diameter that is smaller than the outer diameter of the surgical instrument. In one embodiment, the turbine cleaning wheel is made from a material having a suitable elasticity to allow the surgical instrument to be press fit into the opening 140. The trocar 130 receives washing fluid from a conduit 142 via an input port 144. The washing fluid enters a chamber containing the turbine cleaning wheel 136 causing the turbine cleaning wheel 136 to rotate. It should be appreciated that as the surgical instrument passes through the opening 140, the rotation of the turbine cleaning wheel 136 and the washing fluid cooperate to clean the surgical instrument. The washing fluid is removed via an output port 146 and a conduit 148.

In an embodiment, the trocar 130 further includes a gas port 150 and a conduit 152 that allows removal of gases, such as CO₂ for example. In still another embodiment, a distal cleaning valve 154 may be positioned within the passageway of the cannula 134. The distal cleaning valve 154 includes an aperture or hole 156 that is smaller than the outer diameter of the surgical instrument and having an elasticity that allows the surgical instrument to pass through. As the surgical instrument passes through the hole 156, additional cleaning of the surgical instrument is achieved.

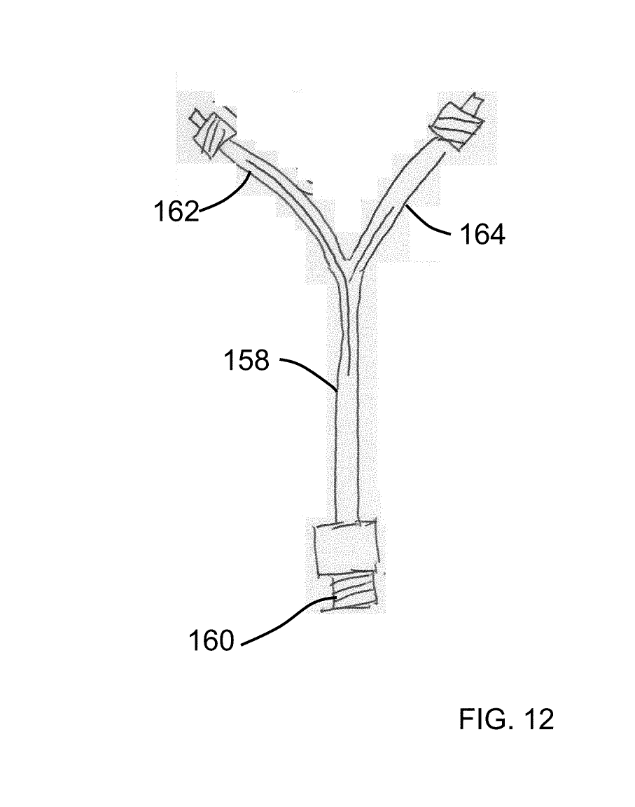

In one embodiment, the trocar 130 may be used with a y-tube conduit 158 shown in FIG. 12. In this embodiment, the conduit 158 includes a first end 160 that couples to the output port 146. The conduit 158 bifurcates into the first conduit 162 and a second conduit 164. The first conduit 162 couples to a washing fluid reservoir (not shown), the second conduit 164 couples to a vacuum or aspiration device (not shown). In one embodiment, the insertion of washing fluid and the aspiration may be performed via a single port or may be performed via two conduits.

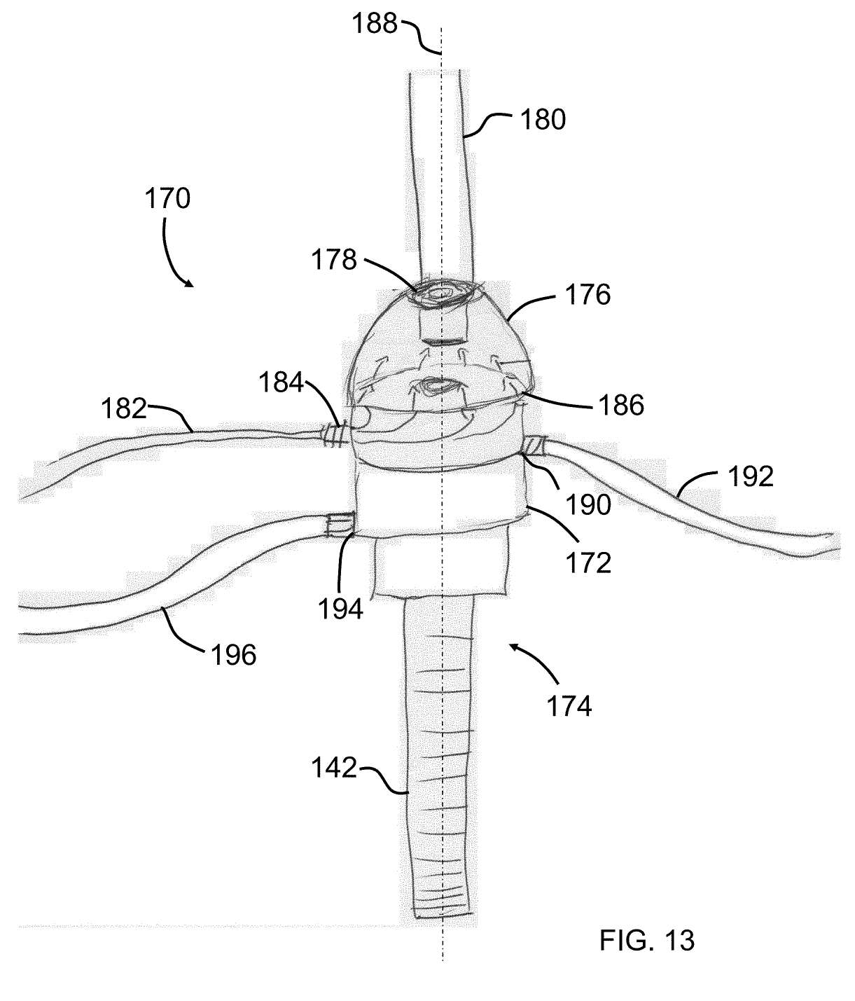

Referring now to FIG. 13, another embodiment is shown of a trocar device 170. The trocar 170 includes a body 172 with a cannula 174 coupled to one end. A dome member 176 having a semi-spherical shape is coupled to the body 172 on an end opposite the cannula 174. In the exemplary embodiment, the dome member 176 is made from a clear, transparent or translucent material. The dome member 176 has a generally hollow interior and an aperture or opening 178. In the exemplary embodiment, the opening 178 is defined by a material that has an elasticity that allows the surgical instrument 180 to pass therethrough with a tight or press fit.

Washing fluid is received via conduit 182 through an input port 184. The input port 184 may be integral with the body 172 or the dome member 176. The input port 184 is fluidly coupled to a plurality of orifices, nozzles or jets 186 that are disposed about one end of the dome member 176. In the exemplary embodiment, the jets 186 are angled inward to direct washing fluid toward a centerline 188 of the trocar 170. It should be appreciated that as the surgical instrument 180 is inserted through the opening 178 (or withdrawn from the patient into the dome member), the washing fluid will contact and clean the surgical instrument 180. It should be appreciated that having the dome member 176 made from a clear material allows the medical personnel to observe the cleanliness state of the surgical instrument 180. An output port 190 and a conduit 192 apply a vacuum to allow the removal of the washing fluid from the dome member 176. A gas port 194 receives a gas, such as CO₂ for example, from a conduit 196.

It should be appreciated that the features of embodiments of FIG. 1, FIG. 10, FIG. 11 and FIG. 13 may be combined and the description of these embodiments is not intended to be limiting. For example, the turbine cleaning wheel 136 of FIG. 11 could be incorporated into the trocar devices 20, 100, 170 for example.

Embodiments disclosed herein provide advantages in allowing a surgical instrument, such as a scope for example, to be cleaned without removing the surgical instrument from a trocar on a patient. It should be appreciated that this improves the efficiency of the surgical procedure reducing time and reducing risks. In the case of a scope, the cleaning may allow the medical personnel improved visual images of the surgical location during the procedure.

The term “about” is intended to include the degree of error associated with measurement of the particular quantity based upon the equipment available at the time of filing the application. For example, “about” can include a range of ±8% or 5%, or 2% of a given value.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, element components, and/or groups thereof.

While the disclosure is provided in detail in connection with only a limited number of embodiments, it should be readily understood that the disclosure is not limited to such disclosed embodiments. Rather, the disclosure can be modified to incorporate any number of variations, alterations, substitutions or equivalent arrangements not heretofore described, but which are commensurate with the spirit and scope of the disclosure. Additionally, while various embodiments of the disclosure have been described, it is to be understood that the exemplary embodiment(s) may include only some of the described exemplary aspects. Accordingly, the disclosure is not to be seen as limited by the foregoing description, but is only limited by the scope of the appended claims.

What is claimed is:

1. A trocar device comprising:
a body having a first passageway sized to receive a surgical instrument;
a cannula coupled to the body, the cannula having a second passageway that cooperates with the first passageway to allow the surgical instrument to pass therethrough;
a plurality of orifices operably coupled to one of the body or the cannula and arranged to direct a washing fluid onto at least a portion of the surgical instrument in one of the first passageway or the second passageway; and
an output port fluidly coupled to receive the washing fluid from one of the first passageway or the second passageway.

2. The trocar device of claim 1, further comprising:
a port member disposed between the body and the cannula, the port member having an input port configured to receive the washing fluid and the output port, the input port being fluidly coupled to a first annular channel; and
wherein the cannula includes a second annular channel, the first annular channel and the second annular channel cooperating to flow the washing fluid to the plurality of orifices.

3. The trocar device of claim 2, wherein the port member further includes a duct fluidly coupled between the output port and the second passageway.

4. The trocar device of claim 3, wherein the port member further includes at least one slot on an end, the at least one slot fluidly coupling the duct to the second passageway.

5. The trocar device of claim 1, wherein the plurality of orifices are disposed on an angle to direct the washing fluid toward a centerline.

6. The trocar device of claim 5, further comprising a dome member coupled to the body, the dome member having an opening on one end sized to receive the surgical instrument, wherein the plurality of orifices are arranged to direct the washing fluid into the dome member.

7. The trocar device of claim 6, wherein the dome member is made from a clear, a transparent or a translucent material.

8. The trocar device of claim 6, wherein the opening is sized to receive the surgical instrument as a press fit.

9. The trocar device of claim 1, further comprising a distal valve coupled within the second passageway, the distal valve having an opening sized to receive the surgical instrument with a press fit.

10. A trocar device comprising:
a body having a first passageway sized to receive a surgical instrument;
a cannula coupled to the body, the cannula having a second passageway that cooperates with the first passageway to allow the surgical instrument to pass therethrough;
a turbine cleaning wheel rotationally coupled to the body, the turbine cleaning wheel having a first opening sized to receive the surgical instrument;
an input port arranged to direct a washing fluid onto the turbine cleaning wheel; and
an output port fluidly coupled to receive the washing fluid from the first passageway.

11. The trocar device of claim 10, further comprising a distal valve coupled within the second passageway, the distal valve having a second opening sized to receive the surgical instrument with a press fit.

12. A method of cleaning a surgical instrument during a surgical procedure, the method comprising:
providing a trocar device in an incision made during the surgical procedure, the trocar device having a body with a first passageway and a cannula with a second passageway;
inserting the surgical instrument into the trocar device;
flowing a washing fluid into the trocar device via an input port;
flowing the washing fluid through a plurality of orifices onto the surgical instrument in one of the first passageway or the second passageway; and
removing the washing fluid from one of the first passageway or second passageway via an output port.

13. The method of claim 12 further comprising:
flowing the washing fluid from the input port into a first annular channel; and
flowing the washing fluid from the first annular channel into a second annular channel to the plurality of orifices.

14. The method of claim 13 further comprising flowing the washing fluid from the second passageway into a duct fluidly coupled to the output port.

15. The method of claim 14 further comprising flowing the washing fluid into the duct via a slot disposed between the duct and the second passageway.

16. The method of claim 12 wherein the orifices are arranged on an angle to flow the washing fluid towards a centerline.

17. The method of claim 12 wherein the plurality of orifices are disposed within a dome member coupled to the body opposite the cannula.

18. The method of claim 17 further comprising inserting the surgical instrument into an opening in the dome member, wherein the opening is sized to receive the surgical instrument as a press fit.

19. The method of claim 12 further comprising moving the surgical instrument through an opening in a distal valve disposed in the cannula, the opening being sized to receive the surgical instrument as a press fit.

20. The method of claim 12 further comprising applying a vacuum to the output port to remove the washing fluid.
